(19) United States
(12) Patent Application Publication
(10) Pub. No.: US 2024/0358494 A1
(43) Pub. Date: Oct. 31, 2024

(54) PERIVASCULAR IMPLANT

(71) Applicant: Universität Heidelberg, Heidelberg (DE)

(72) Inventors: Hannes KENNGOTT, Heidelberg (DE); Thilo HACKERT, Heidelberg (DE); Markus BÜCHLER, Heidelberg (DE); Gabriel Alexander SALG, Heidelberg (DE)

(73) Assignee: Universität Heidelberg, Heidelberg (DE)

(21) Appl. No.: 18/687,769

(22) PCT Filed: Aug. 30, 2022
(86) PCT No.: PCT/EP2022/074035

§ 371 Date: Feb. 28, 2024

(30) Foreign Application Priority Data
Aug. 31, 2021 (EP) 21193913.7

(51) Int. Cl. A61F 2/06; A61B 17/12

(52) U.S. Cl. CPC A61F 2/06; A61B 17/12009

(57) ABSTRACT

The present invention relates to a perivascular implant comprising (i) a housing comprising a receptacle for an effector matrix, and (ii) a fastening element adapted for at least partially embracing a tubular bodily structure, wherein said housing, said receptacle for an effector matrix, and said fastening element are arranged such that after implantation of the device the receptacle for an effector matrix at least partially is in contact with said tubular bodily structure and/or a vasculature originating from said tubular bodily structure, and to uses, methods, and kit related thereto.

The present invention relates to a perivascular implant comprising (i) a housing comprising a receptacle for an effector matrix, and (ii) a fastening element adapted for at least partially embracing a tubular bodily structure, wherein said housing, said receptacle for an effector matrix, and said fastening element are arranged such that after implantation of the device the receptacle for an effector matrix at least partially is in contact with said tubular bodily structure and/or a vasculature originating from said tubular bodily structure, and to uses, methods, and kit related thereto.

Transplantation of islets of Langerhans to selected patients with type 1 and type 3c diabetes mellitus (DM) is an established treatment option. Autologous transplantation can be performed after isolation of islets from the resected pancreas without the need for life-long immunosuppression, whereas islet transplants for patients with type 1 DM rely on allogenic donor islets. Current therapeutic limitations include a shortage of donor material but also a substantial loss of islets and impaired long-term function post transplantation (Salg et al., J Tissue Eng 2019; 10:1-25; Gamble et al., Islets 2018; 10:80-94; Peiris et al., Diabetes 2014; 63:3-11). Scaffold-based tissue engineering approaches extend the range of possible transplantation sites and might present a long-term curative treatment. For a successful translational approach, several requirements and properties of a functional tissue-engineered device have to be considered. The scaffold material itself should not induce cytotoxicity or extensive foreign body response and should preferably support or promote rapid vascularization and the scaffold should be retrievable and possess a certain mechanical strength, at least until tissue remodeling has occurred (Smink et al., Ann Surg 2017; 266:149-157; Siddiqui et al., Mol Biotechnol 2018; 60:506-532).

Previously, it was found that scaffold-based tissue engineering is still hampered by reduced vascularization, causing insufficient nutrition, hypoxia, and immunological host-graft reactions (Salg et al., 2019, loc. cit.). The multitude of studies focusing mostly on aspects of the tissue engineering network have not yet provided structured evidence to define a gold-standard approach. Investigations of a variety of different cells, scaffold materials, fabrication techniques, and transplantation sites have not yet consolidated into an entire process leading towards bioartificial organs.

Thus, there is a need for improved means and methods for implanting cells producing therapeutic compounds into a mammalian host, avoiding at least in part the drawbacks of the prior art. This problem is solved by the means and methods of the present invention, with the features of the independent claims. Preferred embodiments, which might be realized in an isolated fashion or in any arbitrary combination are listed in the dependent claims.

In accordance, the present invention relates to a perivascular implant comprising
(i) a housing comprising a receptacle for an effector matrix, and
(ii) a fastening element adapted for at least partially embracing a tubular bodily structure, wherein said housing, said receptacle for an effector matrix, and said fastening element are arranged such that after implantation of the device the receptacle for an effector matrix at least partially is in contact with said tubular bodily structure and/or a vasculature originating from said tubular bodily structure.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The term “subject”, as used herein, relates to a vertebrate animal, preferably to a mammal. More preferably, the subject is a human, a cattle, a pig, a sheep, a goat, a horse, a cat, a dog, a guinea pig, a mouse, or a rat. Preferably, the subject is a laboratory animal, preferably a guinea pig, a mouse, or a rat. Also preferably, the subject is a livestock, preferably a cattle, a pig, a sheep, a goat, or a horse. Also preferably, the subject is a companion animal, preferably a cat or a dog. Most preferably, the subject is a human.

The term “tubular bodily structure” includes any and all structures in the body of a subject having an overall extended, preferably cylindrical, structure. Preferably, the tubular bodily structure comprises or is a blood vessel or a bundle thereof. Thus, the tubular bodily structure preferably is an artery, a vein, or a structure comprising an artery and/or a vein; the tubular bodily structure may, however, also comprise further structures, such as e.g. a nerve cord. Preferably, the tubular bodily structure comprises or is a medium-size blood vessel, preferably having a diameter of from 2 mm to 2.5 cm, preferably of from 3 mm to 2 cm. The tubular bodily structure preferably is a vascular bundle, in particular an epigastric pedicle. The term “epigastric pedicle” is known to the skilled person. Preferably, the epigastric pedicle is a vascular bundle situated in humans lateral to the navel, typically at a distance of from about 5 cm from the navel.

The term “perivascular” is used herein in a broad sense to relate to a location in the vicinity of a tubular bodily structure, preferably at least one blood vessel. The term includes in particular location in close vicinity to a blood vessel, i.e. directly fastened to a blood vessel; and also a location between two blood vessels, i.e. preferably intervascular location, preferably between an artery and a vein.

The term “implant”, which may also be referred to as “device for implantation”, as used herein, relates to each and every device having the features as specified and being adapted for implantation into a subject. Implantation may be long-term implantation, the term long-term relating to a time frame of at least 4 weeks, preferably at least 2 months, more preferably at least three months, still more preferably at least six months, even more preferably at least one year, most preferably at least two years. There is no principal upper limit to the time an implant can be maintained in the body of a subject; the implant may, however, be rejected and/or experience fouling, which may make revision or removal of the implant necessary. Adaptation for implantation comprises that the device for implantation has an overall size and structure making implantation into the body of a mammalian host possible. As the skilled person understands, the specific size and structure requirement for the implant may vary and depend, among other factors, on the nature of the mammalian host, e.g. its species, size, and/or age, as well as on the intended location of the implant. Preferably, the implant is implanted in a body cavity of the mammalian host, preferably a natural or an artificially created body cavity, more preferably a natural body cavity. Natural body cavities are in particular the abdomen, the peritoneum, the digestive tract, and the lung. Artificial body cavities may be created e.g. in a subcutaneous tissue, a fat tissue, a muscle, the cranium, the pleura, or a breast of the mammalian host. Preferably, adaptation for implantation further comprises not being detrimental to the subject, in particular in cases where the device for implantation is maintained in an implanted state for an extended period of time. Thus, preferably, the device for implantation comprises biocompatible materials, more preferably consists of biocompatible surface materials, most preferably consists of biocompatible materials. Biocompatibility may, in principle, be achieved by adhesion of cells and extracellular material, e.g. matrix material (fouling), frequently followed by encapsulation; or biocompatibility may be achieved by prevention of adhesion of cells and extracellular material.

The implant comprises a housing. The term “housing” is used herein in a broad sense to relate to any structure providing the described properties. Thus, the housing may in principle be made of any material deemed appropriate by the skilled person. Preferably, the housing has a rigidity corresponding to the intended use. Also preferably, the material of the housing is biocompatible. Appropriate materials known in the art and are described e.g. as scaffold materials in Salg et al. (2019, loc. cit.); preferred materials include poly-caprolactone, poly-caprolactame, poly-lactic-co-glycolic acid, poly-lactic acid, poly-glycolic acid, polyurethane, polyvinyl chloride, poly-dioaxanone, poly-acryletherketones such as poly-etheretherketone and poly-etherketoneketone, polyethylene glycol, and polyethylene glycol acrylates such as polyethylene glycol diacrylate and polyethylene glycol dimethacrylate. or mixtures thereof; more preferably, the housing comprises or consists of poly-caprolactone. The housing may have any form deemed appropriate by the skilled person; preferred are overall essentially extended structures providing a length of at least 1 cm, preferably at least 2 cm, more preferably at least 3 cm, along the axis parallel to the tubular bodily structure after implantation. Thus, the housing may have an essentially extended, preferably cylindrical form. The housing, in particular its outer shell preferably has openings allowing in- or outgrowth of tubular bodily structures, in particular blood vessels, in particular at surfaces of the housing contacting the tubular bodily structure. Thus, the housing may comprise channels and/or have at least partially a mesh structure, preferably with openings having an area of from 0.1 mm² to 10 mm². Preferably, the housing is at least partially coated with at least one modulator compound. The term “modulator compound” is used herein in a broad sense to relate to each and every compound modulating the interaction between the housing and the surrounding tissue after implantation. Preferably, said modulator compound is attached covalently to the housing. Preferably, the modulator compound comprises, preferably is, heparin and/or a growth factor, preferably basic fibroblast growth factor (bFGF), nerve growth factor (NGF), and/or vascular endothelial growth factor (VEGF). The above applies to fastening elements, receptacles, bridging element, housing modules, fastening modules, receptacle modules, and bridging modules as specified herein elsewhere mutatis mutandis. Preferably, the effector matrix is inserted into the perivascular implant after its implantation, more preferably after ingrowth, preferably at least one, more preferably at least two weeks, after implantation of the perivascular implant. More preferably, however, the perivascular implant comprises an effector matrix when implanted, i.e. preferably before implantation or during implantation, more preferably at least before the opening in the body used to implant the perivascular implant is closed.

The housing comprises a receptacle for an effector matrix. The term “receptacle” is used herein in abroad sense to relate to each and every component of the housing suitable and/or adapted for receiving at least one effector matrix. Suitable structures depend on the form and structure of the effector matrix to be received. Thus, the receptacle and the effector matrix are preferably adapted to each other in a plug and socket manner. Preferably, the receptacle, as well as optionally the residual housing, are structured such as to provide a large surface, preferably with at least one opening fitting the effector matrix to ensure close contact between the receptacle and the effector matrix. Thus, the receptacle preferably has notches or indentations fitting the effector matrix or parts thereof, wherein said notches or indentations preferably are in open connection to at least one surface of the housing contacting said tubular bodily structure, e.g. so as to allow growth of blood vessels connecting the effector matrix and the tubular bodily structure. The receptacle may, however, also be an opening within the housing into which the effector matrix may be inserted. As will be understood, the latter embodiment may be preferred in cases where it is expected that the effector matrix may have to be exchanged. Preferably, the receptacle comprises a non-cell permeable membrane intervening between the effector matrix and the tubular bodily structure and/or between a vasculature originating from said tubular bodily structure. Also, the perivascular implant may be implanted with a placeholder for an effector matrix to ensure that the planned effector matrix fits into the receptacle after ingrowth of vasculature. More preferably, the receptacle already comprises an effector matrix upon implantation of the perivascular implant.

Preferably, the perivascular implant further comprises an effector matrix. The term “effector matrix”, as used herein, relates to any composition comprising a matrix material and effector cells, both as specified herein below. The effector matrix preferably is adapted to insertion into a pre-implanted perivascular implant or, more preferably, to implantation concomitantly with the perivascular implant; thus, the effector matrix preferably is solid or semisolid. The effector matrix may, also be enclosed in an appropriate enclosure, e.g. a non-cell permeable membrane, a layer of a matrix material not comprising effector cells, and the like. Thus, a liquid, semisolid, or solid effector matrix may be enclosed by an outer layer of matrix material, which may be identical or non-identical to the matrix material of the effector matrix, which outer layer may provide the required stability and/or rigidity for handling. Thus, the effector matrix may form an inner layer comprising effector cells, enclosed by an outer layer not comprising effector cells, wherein said outer layer may be a layer of matrix material not comprising effector cells or a non-cell permeable membrane; such an embodiment is also referred to as a “coated effector matrix” herein. Preferably, the effector matrix is adapted to ensure that the effector cells do not directly contact the tubular bodily structure, tubular bodily structures originating therefrom, and/or other tissues of the subject. Thus, the perivascular implant and/or the effector matrix are preferably adapted to permit removal of all effector cells comprised in the effector matrix from the body of a subject. Preferably, said removal is put into practice by removing the effector matrix only; the removal may, however, also be put into practice by removing the perivascular implant or parts thereof including the effector matrix. The shape of the effector matrix is selected by the skilled person as deemed appropriate, preferably to fit with the receptacle of the perivascular implant. Preferred shapes are those which provide for a high surface area to volume ratio, preferably of at least 2 cm⁻¹, more preferably at least 5 cm⁻¹, still more preferably at least 10 cm⁻¹, most preferably at least 25 cm⁻¹. Thus, the effector matrix may e.g. be provided in the form of one or more rods, which may e.g. be curled or folded to fit into the receptacle, rolled or folded sheets, microbeads, and the like. Preferably, the effector matrix is a bioprinted effector matrix, preferably a 3D-bioprinted effector matrix. Also preferably, the effector matrix is a bioprinted sheet, preferably a folded bioprinted sheet. The effector matrix may, however, also be provided as one or more essentially cylindrical embodiments, which may e.g. be inserted into one or more corresponding channels of the housing. The volume of the effector matrix is essentially determined by the specific application, by the number and type or types of effector cells required, and other factors known to the skilled person. Preferably, the effector matrix has a volume of from 1 mm³ to 5 cm³, more preferably of from 5 mm³ to 1 cm³. As the skilled person understands, the receptacle of the perivascular implant typically will correspond to the effector matrix in volume and shape. The effector matrix comprises at least one type of effector cells; the effector matrix may, however, also comprise additional cells, such as helper cells prolonging survival of effector cells, improving effector cell function, and/or modulating effector cell differentiation; and/or one or more additional type(s) of effector cells.

The term “matrix material” includes each and every material deemed suitable by the skilled person for embedding and maintaining viability of effector cells. Thus, the matrix material is preferably adapted to permit diffusion of nutrients, including e.g. oxygen and glucose, and optionally growth factors to effector cells, and diffusion of an effector compound, such as insulin, from the cells. In accordance, matrix materials forming a semisolid matrix, e.g. a gel, may be used. Also, the matrix material is selected to not be deleterious to the effector cells and/or the subject. Appropriate matrix materials are known in the art, e.g. from Salg et al. (2019), loc. cit. and US 2005/209687 A1. Preferably, the matrix material is selected from alginate, collagen, chitosan, silk fibroin, fibrinogen, gelatin, gelatin methacrylate, polyethylene glycol, polyethylene glycol acrylates, such as polyethylene glycol diacrylate and polyethylene glycol dimethacrylate, or mixtures thereof. Preferably, the matrix material is a material which can be bioprinted, preferably comprises, more preferably consists of, alginate and/or gelatin, more preferably gelatin methacrylate blend hydrogel.

The term “effector cell” is used herein in a broad sense to relate to each and every cell mediating a metabolic and/or physiologic change when implanted into a subject. Preferably, said effect is treatment and/or prevention of disease as specified herein below. Preferably, the effector cell produces or catabolizes a diffusible factor, preferably with a molecular mass of less than 75 kDa, preferably less than 50 kDa, still more preferably less than 25 kDa, most preferably less than 10 kDa. Thus, the effector cell may, in principle, be a cell producing a desirable metabolite, e.g. insulin or coagulation factor IX; and/or catabolizing an undesirable metabolite. Preferably, the effector cells produce a compound having a signaling effect on the subject's metabolism (effector compound). Thus, the effector cells preferably produce a signaling molecule, preferably at least one of insulin, glucagon, a coagulation factor, a growth hormone, and a cytokine. Preferably, the effector cells produce insulin, more preferably human insulin. Thus, the effector cell preferably is an insulin-secreting cell, more preferably a cell secreting human insulin; thus, the effector cells preferably are human beta-cells or human islets of Langerhans. It is, however, also envisaged that the effector cells are cells differentiating into insulin-secreting cells either before integration into the effector matrix, bioprinting and/or implantation (e.g. in vitro); or differentiating into insulin-secreting cells in vivo, e.g. in situ, preferably in the implanted effector matrix. Exemplary effector cells differentiating into insulin-secreting cells include in particular human induced pluripotent stem cells, adipose tissue derived stem cells, mesenchymal stem cells, pancreatic precursor cells, and insulin-transfected cells. Preferably effector cells comprise insulinoma cells. Also preferably, the effector cells comprise cells stably transfected with an expression construct for insulin, preferably human insulin, and optionally endothelial cells. Preferably, the effector cells are comprised in pancreatic islets or pancreatic islet-like clusters.

The term “fastening element”, as used herein, related to any element of a perivascular implant adapted for at least partially embracing a tubular bodily structure. Thus, the fastening element preferably is adapted to hold the perivascular implant in place by providing a tight contact to the tubular bodily structure. The specific conformation of the fastening element is selected by the skilled person based on the specific tubular bodily structure envisaged for fastening, the location of the perivascular implant, and other factors. Preferably, the tubular bodily structure is a blood vessel; thus, the skilled person understands that exerting pressure perpendicular to the longitudinal axis of the blood vessel is undesirable. Thus, the fastening element in such case preferably at least partially embraces the tubular bodily structure, essentially without exerting pressure thereon. More preferably, the fastening element embraces at least half, more preferably at least ¾, most preferably the complete, circumference of the tubular bodily structure. Thus, the fastening element, more preferably the perivascular implant itself, embraces the tubular bodily structure. In such case, the fastening element may be formed by the housing of the perivascular implant, e.g. such that the fastening element is a channel comprised in the housing of the perivascular implant, preferably with a diameter corresponding to the tubular bodily structure.

In the perivascular implant, the housing, the receptacle, and the fastening element are arranged such that after implantation of the device the receptacle for an effector matrix at least partially is in contact with said tubular bodily structure and/or a vasculature originating from said tubular bodily structure. Thus, the perivascular implant is adapted to bring the receptacle in close proximity to the tubular bodily structure in order to allow the receptacle and the tubular bodily structure to interact. At least partial direct contact is preferred, although it may not always be necessary. Preferably, the arrangement is adapted such that outgrowth of tubular bodily structures such as vasculature from the tubular bodily structure at least partially coats the inner lumen of the receptacle, thus allowing exchange of diffusible compounds between the outgrowth and the effector matrix after its implantation. Preferably, the housing, the receptacle and optionally the effector matrix form a channel adapted for at least partially embracing said tubular bodily structure, wherein said channel preferably has a diameter corresponding to the diameter of the tubular bodily structure. Preferably, the receptacle is arranged such that after implantation the effector matrix at least partially is in diffusion contact with said tubular bodily structure and/or a vasculature originating from said tubular bodily structure, wherein said diffusion contact is via interstitial fluid. More preferably, the receptacle is arranged such that after implantation the effector matrix at least partially is in direct contact with said tubular bodily structure and/or a vasculature originating from said tubular bodily structure. It is, however, also envisaged that the fastening element provides one or a multitude of channels mediating growth of vasculature to a receptacle, wherein the receptacle and, after insertion the effector matrix, are not in direct contact with the tubular bodily structure; such indirect contact is also comprised by the term “in contact” as used herein.

The perivascular implant may be a single device. E.g. the perivascular implant may comprise an essentially circular fastening element into which the tubular bodily structure can be inserted, e.g. via an opening in the fastening element or by the fastening element comprising two interconnectable half-circles. The perivascular implant may also be comprised of two half-cylindrical elements together forming a channel, connected e.g. via a hinge, which can be mounted on a tubular bodily structure to embrace said tubular bodily structure. Thus, the fastening element preferably is formed by said housing and/or said receptacle for an effector matrix. It is, however, also envisaged that the perivascular implant may e.g. be comprised of two fastening elements, a housing, and a receptacle; in such case, the housing and the receptacle preferably form a bridging element between said fastening modules, wherein said fastening modules preferably are adapted to be fastened to two non-identical tubular bodily structures, such as to allow a vasculature bridging said two non-identical tubular bodily structures to form. Preferably, the tubular bodily structure at least partially embraced by the first fastening element is an artery and the tubular structure embraced by the second fastening element is a vein, wherein preferably said tubular structures are an artery and a vein of an epigastric pedicle.

The perivascular implant may, however, also be a modular device comprising separate modules; e.g., the fastening element may be a discrete fastening module, with a detachable connection to a receptacle module and/or housing module. The perivascular implant may also be comprised of two half-cylindrical modules together forming a channel, which can be mounted on a tubular bodily structure to embrace said tubular bodily structure. Also, the housing may be a discrete module, with the option to insert one or more receptacle module(s) depending on application. In accordance, the perivascular implant may e.g. be comprised of two fastening modules, a housing module, and a receptacle module; in such case, the housing module and receptacle module preferably form a bridging module intervening said fastening modules, wherein said fastening modules preferably are adapted to be fastened to two non-identical tubular bodily structures, such as to allow a vasculature bridging said two non-identical tubular bodily structures to form. Preferably, the tubular bodily structure at least partially embraced by the first fastening module is an artery and the tubular bodily structure embraced by the second fastening module is a vein, wherein preferably said tubular structures are an artery and a vein of an epigastric pedicle. In the aforesaid embodiment, the bridging module may e.g. be adapted to be connected to said fastening elements via a slot connection which, upon removal, exerts shearing forces on vasculature spanning the modules, such that the bridging element can be removed including any ingrowing vasculature.

Moreover, a first perivascular implant at least partially embracing a first tubular bodily structure may be adapted to be connected via a connector to a second perivascular implant at least partially embracing a second tubular bodily structure. Also, a first perivascular implant may be adapted to be connected to a second perivascular implant on the same tubular bodily structure. In accordance, a second perivascular implant may be arranged along the axis of the tubular bodily structure, or may be arranged in parallel to the axis of the tubular bodily structure. Preferably, the tubular bodily structure at least partially embraced by the first perivascular implant is an artery and the tubular structure embraced by the second perivascular implant is a vein, wherein preferably said tubular structures are an artery and a vein of an epigastric pedicle.

Means and methods for connecting perivascular implant devices of modules thereof are known to the skilled person. Preferred connectors are snap-action connectors, screw-on connectors, or slot connectors

Preferably, the perivascular implant comprises at least one sensor element. The term “sensor element”, as used herein, includes any and all sensing elements deemed appropriate by the skilled person to provide information on a physiological state of a subject or of the state of effector cells comprised in the perivascular implant. Thus, the sensor element may comprise a sensor for a blood and/or physiological parameter, preferably selected from the group consisting of blood glucose, blood insulin, oxygen concentration, carbon dioxide concentration, pH, ATP concentration, NADH concentration, and FADH₂ concentration, more preferably being blood glucose and/or blood insulin. The sensor preferably is selected in accordance with the effector cells comprised or planned to be comprised in the perivascular implant. Preferably, the sensor element comprises at least one of a blood glucose sensor, a blood insulin sensor, an oxygen sensor (e.g. for measuring oxygen consumption rate of effector cells in the effector matrix), a carbon dioxide sensor (e.g. for measuring a carbon dioxide level of effector cells in the effector matrix), a pH sensor (e.g. for measuring pH gradients in effector matrix and/or blood), an ATP sensor (e.g. for measuring ATP levels of effector cells in the effector matrix), an NADH sensor (e.g. for measuring NADH levels of effector cells in the effector matrix), and an FADH₂ sensor (e.g. for measuring FADH₂ levels of effector cells in the effector matrix). Also preferably, the sensor element comprises a blood glucose sensor and/or a blood insulin sensor, in particular in case the effector cells are insulin-secreting cells. Preferably, the glucose sensor is located at the afferent side of the perivascular implant, preferably at the entry side of an artery embraced by the perivascular implant and/or preferably the perivascular implant comprises an insulin sensor located at the efferent side of the perivascular implant, preferably at the exit side of a vein embraced by the perivascular implant. The perivascular implant may, however, also comprise an insulin sensor located at the afferent side of the perivascular implant, preferably at the entry side of a vein embraced by the perivascular implant. Also preferably, the sensor element comprises an oxygen sensor, a glucose sensor, a pH-sensor, a lactate sensor, an insulin sensor, a C-peptide sensor, an inflammatory biomarker sensor, and/or a sensor for a marker of cell viability, any of which may preferably be comprised in or in the vicinity of the receptacle. Appropriate sensors are known in the art; in an embodiment, the sensor element is a microsensor, in a further embodiment an enzymatic microsensor.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50%, at least 60%, at least 70%, at least 80%, or at least 90% of the subjects of a given cohort or population. Preferably, treating is treating diabetes, preferably diabetes type 1 (preferably including MODY diabetes), diabetes type 2, diabetes type 3, primary hypothyroidism, pituitary insufficiency, adrenal insufficiency, secondary ovarial insufficiency (such as e.g., Sheehan syndrome), primary hypogonadism, hypogonadotropic male infertility, as a form of gender reassignment hormone therapy, or intrinsic or extrinsic or combined coagulation system disorder (such as e.g., von-Willebrand-Jürgens syndrome). More preferably, treating is treating diabetes, preferably diabetes type 1 (preferably including MODY diabetes), diabetes type 2, or diabetes type 3.

The term “preventing”, as used herein, relates to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from metastasis, invasion, and/or remission. Preferably, a cohort or population of subjects is envisaged in this context which normally, i.e. without preventive measures according to the present invention, would develop at least one of metastasis, invasion, and/or remission. Whether a portion is statistically significant can be determined without further ado by the person skilled in the art using various well known statistic evaluation tools discussed elsewhere in this specification.

Advantageously, it was found in the work underlying the present invention that by coating a housing of a perivascular implant with compounds mediating and/or improving ingrowth of vasculature, supply of effector cells with factors required for their maintenance can be improved; moreover, it was found that by bringing an implant into close vicinity of a blood vessel, ingrowth of vasculature can be improved, in particular in case the implant is implanted between an artery and a vein.

The definitions made above apply mutatis mutandis to the following. Additional definitions and explanations made further below also apply for all embodiments described in this specification mutatis mutandis.

The present invention further relates to effector cells for treatment of disease, preferably diabetes, wherein said effector cells are comprised in a perivascular implant according to the present invention; preferably, said disease is diabetes.

The present invention also relates to a method for treating disease, preferably diabetes, in a subject comprising implanting a perivascular implant according to the present invention into said subject.

The method of treating of the present invention, preferably, is an in vivo method. Moreover, it may comprise steps in addition to those explicitly mentioned above. For example, further steps may relate, e.g., to diagnosing disease before treatment, or surveilling the perivascular implant and/or the subject during treatment. Moreover, one or more of said steps may be performed by automated equipment. Preferably, the method comprises implanting the perivascular device to embrace a tubular bodily structure.

The present invention also relates to a kit comprising a (i) at least two modules of a modular perivascular implant of the present invention and/or (ii) a perivascular implant and at least one of an effector matrix of the present invention, a second perivascular implant, and a bridging unit of the present invention.

The term “kit”, as used herein, refers to a collection of the aforementioned compounds, means or reagents which may or may not be packaged together. The components of the kit may be comprised as separate modules as specified herein above or provided as a single perivascular implant. An optional packaging of the kit in an embodiment allows translocation of the compounds of the kit, in particular common translocation; thus, the packaging may in particular be a transportable container comprising all specified components. Moreover, it is to be understood that the kit of the present invention may be used for practicing the methods referred to herein above. It is, in an embodiment, envisaged that all components are provided in a ready-to-use manner for practicing the methods referred to above. Further, the kit preferably contains instructions for carrying out said methods. The instructions can be provided by a user's manual in paper- or electronic form. For example, the manual may comprise instructions for interpreting the results obtained when carrying out the aforementioned methods using the kit. Preferably, the kit comprises further components. Preferably, the kit is adapted for use in a method of the present invention, more preferably is adapted to comprise all components required to perform said method or methods.

The present invention also relates to a method of producing a perivascular implant, comprising
(a) producing a perivascular implant as specified herein above; and
(b) producing an effector matrix of the present invention,
   wherein said housing and said effector matrix are produced to form a central channel adapted for embracing a tubular bodily structure of a subject. Preferably, said method comprises further step (a0) determining the diameter and/or geometry of a tubular bodily structure at a planned implantation site, and preferably further comprises adjusting the diameter and/or geometry of said central channel to the diameter of a tubular bodily structure determined in step (a0). In accordance, the perivascular implant preferably is adapted to the anatomy of the subject.

Preferably, the housing, the effector matrix, and/or the matrix material is/are biocompatible as specified herein above. Also preferably, the effector matrix, the housing, and/or the fastening element is/are not in liquid communication with a subject's circulatory system at the time of implantation, more preferably at the time of and after implantation. Also preferably, the effector matrix, and optionally the perivascular implant, can be explanted and resected without leaving effector cells within the body of the subject.

In view of the above, the following embodiments are particularly envisaged:
Embodiment 1: A perivascular implant comprising
(i) a housing comprising a receptacle for an effector matrix, and
(ii) a fastening element adapted for at least partially embracing a tubular bodily structure, wherein said housing, said receptacle for an effector matrix, and said fastening element are arranged such that after implantation of the device the receptacle for an effector matrix at least partially is in contact with said tubular bodily structure and/or a vasculature originating from said tubular bodily structure.
Embodiment 2: The perivascular implant of embodiment 1, wherein said tubular bodily structure comprises at least one blood vessel.
Embodiment 3: The perivascular implant of embodiment 1 or 2, wherein said tubular bodily structure comprises at least one blood vessel, preferably a medium-size blood vessel, preferably having a diameter of from 2 mm to 25 mm.
Embodiment 4: The perivascular implant of any one of embodiments 1 to 3, wherein said tubular bodily structure further comprises at least one nerve, preferably a nerve cord.
Embodiment 5: The perivascular implant of any one of embodiments 1 to 4, wherein said tubular bodily structure is a vascular bundle, preferably an epigastric pedicle.
Embodiment 6: The perivascular implant of any one of embodiments 1 to 5, wherein said fastening element is formed by said housing and/or said receptacle for an effector matrix.
Embodiment 7: The perivascular implant of any one of embodiments 1 to 6, wherein said perivascular implant further comprises an effector matrix.
Embodiment 8: The perivascular implant of any one of embodiments 1 to 7, wherein said housing, said receptacle for an effector matrix and optionally said effector matrix form a channel adapted for at least partially embracing said tubular bodily structure.
Embodiment 9: The perivascular implant of any one of embodiments 1 to 8, wherein said channel has a diameter corresponding to the diameter of the tubular bodily structure.
Embodiment 10: The perivascular implant of any one of embodiments 1 to 9, wherein said housing has an essentially cylindrical form.
Embodiment 11: The perivascular implant of any one of embodiments 1 to 10, wherein said housing has a mesh structure, preferably with openings having an area of from 0.1 mm2 to 10 mm2.
Embodiment 12: The perivascular implant of any one of embodiments 1 to 11, wherein said housing comprises, preferably consists of, poly-caprolactone.
Embodiment 13: The perivascular implant of any one of embodiments 1 to 12, wherein said housing is at least partially coated with at least one modulator compound.
Embodiment 14: The perivascular implant of embodiment 13, wherein said modulator compound is heparin and/or a growth factor, preferably basic fibroblast growth factor (bFGF), nerve growth factor (NGF), and/or vascular endothelial growth factor (VEGF).
Embodiment 15: The perivascular implant of any one of embodiments 1 to 14, wherein said implant is comprised of two essentially half-cylindrical elements together forming a central channel enclosing said tubular bodily structure.
Embodiment 16: The perivascular implant of embodiment 15, wherein said two essentially half-cylindrical elements are connectable by a snap-action connection.
Embodiment 17: The perivascular implant of any one of embodiments 1 to 16, wherein said perivascular implant is adapted to embrace said tubular bodily structure after implantation.
Embodiment 18: The perivascular implant of any one of embodiments 1 to 17, wherein said perivascular implant is a snap-on implant.
Embodiment 19: The perivascular implant of any one of embodiments 1 to 18, wherein said perivascular implant is a first perivascular implant and further comprises a connector permitting interconnection to a second perivascular implant.
Embodiment 20: The perivascular implant of embodiment 19, wherein said second perivascular implant is arranged along the axis of the tubular bodily structure.
Embodiment 21: The perivascular implant of embodiment 19 or 20, wherein said second perivascular implant is arranged in parallel to the axis of the tubular bodily structure.
Embodiment 22: The perivascular implant of any one of embodiments 19 to 21, wherein said second perivascular implant at least partially embraces a second tubular bodily structure.
Embodiment 23: The perivascular implant of any one of embodiments 19 to 22, wherein the tubular bodily structure at least partially embraced by the first perivascular implant is an artery and wherein the tubular structure embraced by the second perivascular implant is a vein.
Embodiment 24: The perivascular implant of any one of embodiments 19 to 23, wherein said tubular structures are an artery and a vein of an epigastric pedicle.
Embodiment 25: The perivascular implant of any one of embodiments 19 to 24, wherein said first perivascular implant and said second perivascular implant are connected via a bridging element.
Embodiment 26: The perivascular implant of any one of embodiments 19 to 25, wherein a first fastening element at least partially embracing a first tubular bodily structure and a second fastening element at least partially embracing a second tubular bodily structure are connected via a bridging element.
Embodiment 27: The perivascular implant of embodiment 26, wherein said bridging element at least partially has a mesh structure, preferably with openings having an area of from 0.1 mm2 to 10 mm2.
Embodiment 28: The perivascular implant of embodiment 26 or 27, wherein said bridging element comprises, preferably consists of, poly-caprolactone.
Embodiment 29: The perivascular implant of any one of embodiments 26 to 28, wherein said bridging element is at least partially coated with at least one modulator compound.
Embodiment 30: The perivascular implant of any one of embodiments 19 to 29, wherein said first perivascular implant, said second perivascular implant, and optionally said bridging element are provided as a single device.
Embodiment 31: The perivascular implant of embodiment 19, wherein the perivascular implant and the second perivascular implant are arranged after interconnection along an axis formed by the tubular bodily structure.
Embodiment 32: The perivascular implant of any one of embodiments 8 to 31, wherein the diameter of said internal channel and/or the number of effector cells is adjusted in a patient-specific manner.
Embodiment 33: The perivascular implant of any one of embodiments 1 to 32, wherein said perivascular implant comprises at least one sensor element.
Embodiment 34: The perivascular implant of embodiment 33, wherein said sensor element comprises sensor for a blood and/or physiological parameter.
Embodiment 35: The perivascular implant of embodiment 34, wherein said blood and/or physiological parameter is selected from the group consisting of blood glucose, blood insulin, oxygen concentration, carbon dioxide concentration, pH, ATP concentration, NADH concentration, and FADH2 concentration, preferably is blood glucose and/or blood insulin.
Embodiment 36: The perivascular implant of any one of embodiments 1 to 35, wherein said perivascular implant comprises a glucose sensor located at the afferent side of the perivascular implant, preferably at the entry side of an artery embraced by the perivascular implant.
Embodiment 37: The perivascular implant of any one of embodiments 1 to 36, wherein said perivascular implant comprises an insulin sensor located at the efferent side of the perivascular implant, preferably at the exit side of a vein embraced by the perivascular implant.
Embodiment 38: The perivascular implant of any one of embodiments 1 to 37, wherein said perivascular implant comprises an insulin sensor located at the afferent side of the perivascular implant, preferably at the entry side of a vein embraced by the perivascular implant.
Embodiment 39: The perivascular implant of any one of embodiments 1 to 38, wherein said perivascular implant further comprises an effector matrix.
Embodiment 40: The perivascular implant of any one of embodiments 1 to 39, wherein said effector matrix comprises a matrix material and effector cells.
Embodiment 41: The perivascular implant of any one of embodiments 1 to 40, wherein said effector matrix is a bioprinted effector matrix, preferably a 3D-bioprinted effector matrix.
Embodiment 42: The perivascular implant of any one of embodiments 1 to 41, wherein said matrix material comprises, preferably consists of, gelatin methacrylate blend hydrogel.
Embodiment 43: The perivascular implant of any one of embodiments 40 to 42, wherein said effector cells secrete at least one of insulin, glucagon, a growth hormone, and a cytokine.
Embodiment 44: The perivascular implant of any one of embodiments 40 to 43, wherein said effector cells comprise insulin-secreting cells, preferably stably transfected with an expression construct for human insulin, and optionally endothelial cells.
Embodiment 45: The perivascular implant of any one of embodiments 1 to 44, wherein said effector matrix has a high surface area to volume ratio, preferably of at least 2 cm-1, more preferably at least 5 cm-1, still more preferably at least 10 cm-1, most preferably at least 25 cm-1.
Embodiment 46: The perivascular implant of any one of embodiments 1 to 45, wherein said effector matrix comprises an inner layer comprising effector cells and an outer layer not comprising effector cells.
Embodiment 47: The perivascular implant of any one of embodiments 1 to 46, wherein said receptacle comprises a non-cell permeable membrane intervening between the effector matrix and the tubular bodily structure and/or a vasculature originating from said tubular bodily structure.
Embodiment 48: The perivascular implant of any one of embodiments 1 to 47, wherein said effector matrix is enclosed by a non-cell permeable membrane.
Embodiment 49: Effector cells for treatment of disease, preferably diabetes, wherein said effector cells are comprised in a perivascular implant according to any one of embodiments 1 to 48.
Embodiment 50: A method for treating disease, preferably diabetes, in a subject comprising implanting a according to any one of embodiments 1 to 48 into said subject.
Embodiment 51: The method of embodiment 50 wherein said implanting comprises implanting said perivascular device to embrace a tubular bodily structure.
Embodiment 52: A kit comprising a (i) at least two modules of a modular perivascular implant according to any one of embodiments 1 to 48 and/or (ii) a perivascular implant and at least one of an effector matrix, a second perivascular implant, and a bridging unit.
Embodiment 53: A method of producing a perivascular implant, comprising
(a) producing a perivascular implant as specified in any one of embodiments 1 to 48; and
(b) producing an effector matrix,
   wherein said housing and said effector matrix are produced to form a central channel adapted for embracing a tubular bodily structure of a subject.
Embodiment 54: The method of embodiment 53, wherein said method comprises further step (a0) determining the diameter and/or geometry of a tubular bodily structure at a planned implantation site, and wherein said method further comprises adjusting the diameter and/or geometry of said central channel to the diameter of a tubular bodily structure determined in step (a0).
Embodiment 55: The subject matter of any of the preceding embodiments, wherein said housing, said effector matrix, and/or said matrix material is/are biocompatible.
Embodiment 56: The subject matter of any of the preceding embodiments, wherein said effector matrix, said housing, and/or said fastening element is/are not in liquid communication with a subject's circulatory system at the time of implantation.
Embodiment 57: The subject matter of any of the preceding embodiments, wherein said subject is a mammal, preferably a human.
Embodiment 58. The subject matter of any of the preceding embodiments, wherein said effector matrix, and optionally the perivascular implant, can be explanted and resected without leaving effector cells within the body of the subject.

All references cited in this specification are herewith incorporated by reference with respect to their entire disclosure content and the disclosure content specifically mentioned in this specification.

### FIGURE LEGENDS

FIG. 1: Exemplary embodiment of a perivascular implant 100 with a housing 110, a receptacle 120 and an effector matrix 130, embracing a tubular bodily structure 140; in the exemplary embodiment, a fastening element 160 is formed by the housing 110 as a central channel of the perivascular implant 100.

FIG. 2: Side view (a, b), inclined top view (c), and open side view of the exemplary embodiment of FIG. 1.

FIG. 3: Exemplary embodiment of an effector matrix 130 as a folded sheet which can be bioprinted.

FIG. 4: (a) Exemplary embodiment of a perivascular implant 100 comprising two fastening elements 160 embracing an artery 140 (right) and a vein 140 (left) respectively, connected by a bridging element 150 and comprising two sensor elements 170; (b) cross sectional view along axis A-A′.

FIG. 5: Cross-sectional view of an exemplary embodiment comprising two perivascular implants 100 embracing an artery 140 (right) and a vein 140 (left) respectively, partially coated with a growth factor coating 180, e.g. VEGF.

FIG. 6: Automated identification of vascular structures and analysis of total vessel network length of housing component 110 (here: 3D-printed PCL scaffold) after explantation from fertilized chick egg. The heparin-functionalized housing component 110 was biocompatible. Extensive vascular ingrowth and neoangiogenesis was achieved within the assay period of 9 days.

FIG. 7: Vascular ingrowth and neoangiogenesis into and around the housing component 110 are enhanced once the surface of the housing component was functionalized by covalent binding of heparin.

FIG. 8: FIG. 3: Chorioallantoic membrane assay is a suitable model for investigating angiogenesis in tissue-engineered grafts. Extensive, rapid vascular ingrowth is seen in both PCL housing component and cell-containing hydrogel effector matrix structure after the 9-day assay period. Ex ovo CAM assay experiments enabled direct comparison of heparinized PCL housing with untreated controls and validated the beneficial properties of heparinization for enhanced vascular ingrowth (a-c, a: arrow indicates eye of chicken embryo). In ovo CAM assay experiments were used for investigation of 3D-bioprinted effector cell-containing effector matrix (d-f, d: arrow indicates effector matrix). Vascular structures (arrows) penetrated into the scaffold (g, h). (g) Anti-insulin immunohistochemical staining of CAM assay explant. (h) H&E staining of CAM assay explant. Rapid vascularization maintained viability and function of insulin-secreting effector cells. Peri- and intra-insular (asterisk) vessels were detected (g, h). Scale bar (a, d) 10 mm, (b, c, e, f) 2 mm, (g, h) 50 μm. (color, double column).

The following Examples shall merely illustrate the invention. They shall not be construed, whatsoever, to limit the scope of the invention. Data relating to the Examples below were also published in Salg et al. (2022), J Tissue Eng 13, 20417314221091033, doi: 10.1177/20417314221091033, which is herewith incorporated by reference.

### EXAMPLE 1: EXEMPLARY DESCRIPTION OF MANUFACTURING A PERIVASCULAR IMPLANT 100 COMPRISING A HOUSING 110, A FASTENING ELEMENT 160 AND EFFECTOR MATRIX 130

1.1 Computer-Aided Design (CAD) Model Creation and Slicing for Fabrication by d-Printing or 3D-Bioprinting

CAD models of the housing 110, fastening element 160 and effector matrix 130 structures were created using the open-source package Blender (www.blender.org). The models were converted from Standard Triangulation Language to numerical control G programming language using the Cura software package (v4.1, Ultimaker, Utrecht, NL; available from www.ultimaker.com/en/products/ultimaker-cura-software) for dual-extrusion 3D printing of the housing 110 and fastening element 160 consisting of polycaprolactone. For 3D bioprinting of the effector matrix 130 an integrated slicing software was applied (CellInk, Gothenburg, Sweden).

### 3D-Printing of Housing 110 and Fastening Element 160 Consisting of Polycaprolactone, Heparin Surface Functionalization Thereof and Subsequent Growth Factor Addition.

Housing 110 and fastening element 160 components were fabricated using a dual-extrusion-based 3D printer (UM S5, Ultimaker, Utrecht, Netherlands). Polycaprolactone filament (Facilan™ PCL 100 Filament 2.85 mm, 3D4Makers, Haarlem, Netherlands; MW: 50 000 g/mol) was used for these structures and polyvinyl alcohol filament (PVA; Ultimaker) as a sacrificial, water-soluble support structure. Polycaprolactone was extruded with an AA 0.25 mm, PVA with a BB 0.4 mm print head, using the following settings: print speed 20 mm/s, build plate temperature 30° C., fan speed 100%, AA print head temperature 140° C., BB print head temperature 215° C. In order to print polycaprolactone at temperatures as low as 140° C., the g-code was manually edited by prefix code ‘M302’ to avoid device-specific conformity checks. For heparin surface functionalization, 1% (w/v) heparin (Lot #H0200000, Merck, Darmstadt, Germany) was dissolved in 0.05 m 2-(N-morpholino) ethanesulfonic acid monohydrate (MES) buffer (Lot #K49565026903, Merck) at a pH of 5.5. Quantities of 0.5 m 1-ethyl-3-(3-dimethylaminopropyl) carbodiimide (EDC) (Lot #E7750, Merck) and 0.5 m N-hydroxysuccinimide (NHS) (Lot #BCBW6640, Merck) were added to the heparin solution. 3D-printed housing 110 and fastening elements 160 were previously equilibrated for 30 min in MES buffer and subsequently immersed in reaction mixture. The reaction mixture was then stirred for 8 h at room temperature. The reaction was stopped by extensive washing with sterile H₂O to remove unbound heparin. Growth factor addition was performed by immersion of scaffolds in beta fibroblast growth factor (bFGF; 500 ng/ml) or nerve growth factor (NGF; 500 ng/ml) in phosphate buffered saline (PBS) for 2 h at room temperature. Elements could be stored in PBS. Covalent binding of heparin on polycaprolactone was proved by scanning electron microscopy compared with untreated controls. Functionalization remained stable over time (4 weeks, storage in PBS).

### 1.2 3D-Bioprinting of Effector Cell-Containing Effector Matrix 130 Using an Insulin-Secreting Cell Line or a Co-Culture of Insulin-Secreting Effector Cells and Additional Helper Cells

The following exemplary protocol for an experimental setup was used for the INS-1 832/3 cell line as insulin-secreting effector cells (obtained from Merck, Darmstadt, Germany) and a HUVEC cell line as additional helper cells (co-culture; obtained from the American Type Culture Collection, Manassas, VA, USA). Mycoplasma testing was performed monthly by polymerase chain reaction. Insulin-secreting effector cells were used until passage 10; insulin-producing function was ensured by selection through Geneticin resistance. Effector cells were cultivated in RPMI-1640 (Gibco, Thermo Fisher Scientific, Waltham, MA, USA) supplemented with 10% fetal bovine serum (Gibco), 1% Geneticin (Merck), 1% HEPES IM (Gibco), 1% sodium pyruvate 100 mM (Merck), and 0.1% 2-mercaptoethanol (Merck). Helper cells were cultivated in endothelial cell growth medium (Lot #211-500, Cell Applications, San Diego, CA, USA) supplemented with 1% penicillin/streptomycin (Merck) and 5% fetal bovine serum. For co-cultures, culture medium composition was chosen according to cell ratio. Cells were grown in T75 flasks (Falcon®, Corning, NY, USA) at 37° C. and 5% CO₂.

Bioprinting was performed using the 3D-bioprinter BioX (CellInk, Gothenburg, Sweden). Pneumatic extrusion print heads were used for extrusion of matrix material, thereafter called bioink. 3×10⁶ cells/ml effector matrix material were used for bioprinting. The cells, either insulin-secreting cells (here: INS-1 832/3) only or insulin-secreting cells with endothelial cells (here: HUVEC) in 1:2 ratio, were diluted in either RPMI-1640 or a 1:2 mixture of RPMI-1640 and endothelial cell growth medium, respectively, and gently mixed 1:10 with a gelatin methacrylate effector matrix additionally containing alginate, xanthan gum and laminin 411 (GelXA LAMININK 411, Lot #IK-3X2123, CellInk) using female-female Luer-lock-adapted syringes. The insulin-secreting cell/endothelial cell ratio was chosen based on the natural islet microenvironment and, due to superior results, compared with a 1:5 ratio. The cell-laden effector matrix was transferred to a UV-shielded cartridge and centrifuged at 100 g for 1 min to remove any air. The cartridge (pre-cooled to 4° C.) was loaded into pneumatic print heads. Bioprinting in 24-well plates (Falcon®, Corning) was performed with the following settings: 21-gauge conical nozzle, extrusion pressure 23 kPa, print speed 8 mm/s, 50 ms pre-flow delay, infill 15%, 2 s crosslinking at 405 nm with 5 cm distance to printed layer.

### EXAMPLE 2: EXEMPLARY DESCRIPTION OF FUNCTIONALITY TESTING: GLUCOSE-STIMULATED INSULIN SECRETION (GSIS)

For GSIS experiments, insulin-secreting effector cells were stained with red fluorescent membrane inserting dye PKH-26 (Lot #SLBW0232, Merck) according to the manufacturer's protocol prior to mixing with the effector matrix for bioprinting. In brief, cells were trypsinized using 0.25% Trypsin-EDTA (Gibco), rinsed with Dulbecco's PBS (DPBS; PromoCell GmbH, Heidelberg, Germany), and finally pelleted. The pellet was resuspended in Diluent A, and PKH-26 dye dissolved in Diluent A was added to the cells. After rapid mixing and incubation, culture medium was added. The cell suspension was centrifuged and further washing steps were performed. The insulin secretion of 3D-bioprinted effector cells in effector matrix 130 (low glucose: n=22; high glucose: n=20) and effector cell and helper cell co-culture in effector matrix (low glucose: n=22; high glucose: n=21) groups, effector cells seeded on PCL/heparin-PCL housing elements (2×10⁵ cells in 1 ml RPMI-1640 in per well), and the 2D monolayer control group was measured. In the 2D monolayer culture group, effector cells were seeded in 4-well chamber slides (10\hat{ }5 cells in 1 ml RPMI-1640 per well) (Nunc® Lab-Tek®, Thermo Fisher Scientific). The medium was changed after 2 days, and GSIS was performed on day 3 in all conditions. For preparation of the GSIS solution, SILAC RPMI-1640 Flex (A2494201, Gibco) was supplemented with MgSO4 (1.16 mmol/l end concentration) (Merck), CaCl₂) (2.5 mmol/l end concentration) (Merck), 20 mM HEPES, and 0.2% BSA (Merck). GSIS was initiated by rinsing the cells once with low-glucose solution (1.67 mM D-glucose), followed by incubation for 1 h in 1 ml low-glucose solution. After that, either 1 ml of low-glucose solution or 1 ml of high glucose solution (16.7 mM D-glucose) was added, followed by incubation for 2 h. A quantity of 500 μl medium was taken and briefly spun down in a 1.5-ml Eppendorf tube. Next, 400 μl supernatant was used for determination of insulin concentration by chemiluminescence immunoassay (ADVIA CENTAUR, Siemens Medical Solutions, Malvern, PA, USA). After GSIS of 2D samples on chamber slides, cells were incubated in 5% formaldehyde solution for 15 min, rinsed with DPBS twice, dried for 10 min, and covered with Fluoroshield Mounting Medium with 4′,6-diamidino-2-phenylindole (DAPI; Abcam, Cambridge, UK) and a coverslip. Similarly, 3D-bioprinted samples were fixated and transferred to a glass slide, covered with two drops of Shandon Consul mounting medium (Thermo Fisher Scientific), and squashed with a coverslip until flattened. Cells were counted using a Leica DMi8 fluorescence microscope with the following settings for PKH-26 imaging: 10× magnification, Y3 filter block, 260 ms exposure time, gain 7. DAPI imaging was performed with the following settings: 10× magnification, DAPI filter block, 10.5 ms exposure time, gain 4. Image processing was performed using Leica LAS X software, and subimages were assembled to mosaics depicting whole domes or whole well bottoms. Cells were counted using ImageJ (Fiji package). In the case of PCL-polymer housing-based culture, cells were lysed using radioimmunoprecipitation assay (RIPA) buffer supplemented with protease inhibitor (complete Mini, Roche, Basel, Switzerland) and incubated on ice for 10 min. Protein concentration was determined using a bicinchoninic acid (BCA) assay (Pierce BCA Protein Assay Kit, Thermo Fisher Scientific). The assay was performed according to the manufacturer's protocol. In n=12 wells of a 24-well plate, 10\hat{ }5 INS-1 cells were seeded in 1 ml RPMI-1640 for correlation of total protein to cell number. After 48 h the medium was changed, followed by another 24 h of incubation. Cells were lysed using 250 μl RIPA buffer+protease inhibitor in n=6 wells and total protein was determined. The residual wells were fixed with 5% formalin, rinsed twice with PBS, and mounted using Fluoroshield Mounting Medium with DAPI. After cell counting, a conversion factor between cell number and total protein was obtained.

### EXAMPLE 3: VASCULARIZATION OF PERIVASCULAR IMPLANTS: XENOTRANSPLANTATION OF PERIVASCULAR IMPLANTS 100 USING A HOUSING COMPONENT 110 AND EFFECTOR MATRIX COMPONENT 130 ONTO THE CHORIOALLANTOIC MEMBRANE OF FERTILIZED CHICKEN EGGS RESULTED IN EXTENSIVE VASCULARIZATION THAT ENABLED MAINTENANCE OF VIABILITY AND FUNCTION OF EFFECTOR CELLS

As described before (Zhao et al., Int J Cancer 2018; 142:1440-52, Int J Cancer), fertilized eggs from genetically identical hybrid Lohman Brown chickens were obtained from a local ecological hatchery (Gefluegelzucht Hockenberger, Eppingen, Germany). Eggs were delivered at day 0 of chick development and were immediately cleaned with 70% warm ethanol. The eggs were placed in a digital motor breeder (Type 168/D, Siepmann GmbH, Herdecke, Germany) at 37.8° C. and 45-55% humidity with an activated turning mechanism to start day 1 of the embryonic chick development. Four days after incubation, the turning mechanism of the incubator was switched off and a small hole was cut into the eggshell to detach the embryonic structures from the eggshell by removing 3 ml albumin. The hole was covered with Leukosilk® tape (BSN medical, Hamburg, Germany), and the eggs were incubated further with the turning mechanism switched off. On day 9 of embryonic development, the tape was removed and the epithelial layer of the chorioallantoic membrane (CAM) was gently scratched with a syringe needle to ensure immediate blood supply to the xenotransplant or perivascular implant 100, respectively. Housing component 110 and bioprinted effector matrix component 130 containing effector cells (here: xenotransplant) were placed on the CAM. The housing component 110 consisted of 3D-printed PCL scaffolds functionalized with covalently bound heparin and plain PCL scaffolds as described in Example 1. Prior to implantation, housing components 110 were sterilized with 70% ethanol for 48 h. For explantation, the chicks were ethically euthanized at day 18 of development, 3 days before hatching, as described before (Aleksandrowicz et al., ALTEX 2015; 32:143-147). Housing component 110 and bioprinted effector matrix 130 containing effector cells were excised including the surrounding CAM and briefly washed in PBS before further imaging. Each specimen was imaged by stereomicroscopy (Leica MZ10 F, Leica Microsystems GmbH, Wetzlar, Germany). Images of housing components were analyzed using an automatic image analysis software (WimCAM; CAM Assay Image Analysis Solution, Release 1.1, Wimasis, 2016). Excised effector matrix components 130 were fixated in 5% formaldehyde (Otto Fischar GmbH & Co. KG, Saarbruecken, Germany) and transferred to 70% ethanol after 24 h. The fixated, explanted effector matrices 130 were embedded using HistoGel™ (Lot #370234, HG-4000-012, Thermo Fisher Scientific) and cryomolds (Tissue-Tek™, Cryomold™, Thermo Fisher Scientific) according to the manufacturers' instructions. After paraffin embedding, randomly chosen blocks from each experimental group were continuously sampled in 5 μm serial sections, numbered, and processed for histology. Slides with odd numbers were stained with Mayer's Hematoxylin-Eosin (H/E), while those with even numbers were immunostained for insulin. Therefore, a primary anti-insulin antibody (monoclonal mouse IgG, 2D11-H5, Lot #SC-8033, SantaCruz, Dallas, TX, USA), overnight 1:100 in background reducing antibody diluent (S3022, Dako, Agilent Tech., Santa Clara, CA, USA), and a polyclonal goat anti mouse secondary antibody (Dako, Agilent Tech.), 3-3′diaminobenzidine staining with subsequent hematoxylin counter-staining, were used. In addition, randomly chosen samples were immunostained for endothelial and endothelial progenitor cells with a primary anti-chicken CD34 antibody (monoclonal mouse IgG; Lot #AV138, UniProt E1BUT3, Avian Immunology toolbox project, Bio-Rad Laboratories GmbH, Feldkirchen, Germany) to identify newly formed vascular structures in the CAM assay. Whole slides were scanned at 40× magnification using a NanoZoomer S60 Digital Slide Scanner (Hamamatsu Photonics, Hamamatsu City, Japan). Stained tissue slides were analyzed using the ilastik software package (Berg et al., Nat Methods 2019; 16:1226-32) for supervised machine learning (ilastik: interactive machine learning for [bio]image analysis, v1.3.3, open-source, www.ilastik.org/download.html). Insulin-secreting islets, here presenting the effector cells, were segmented using the pixel classification workflow (islet, non-islet, background [not islet, not non islet]). First, a random forest classifier was trained manually, and subsequent batch processing was performed. Due to limitations of the machine-learning strategy in differentiating xenograft and CAM tissue, the xenograft area was determined using ImageJ (Fiji package described by Schindelin et al., Nat Methods 2012; 9:676-682).

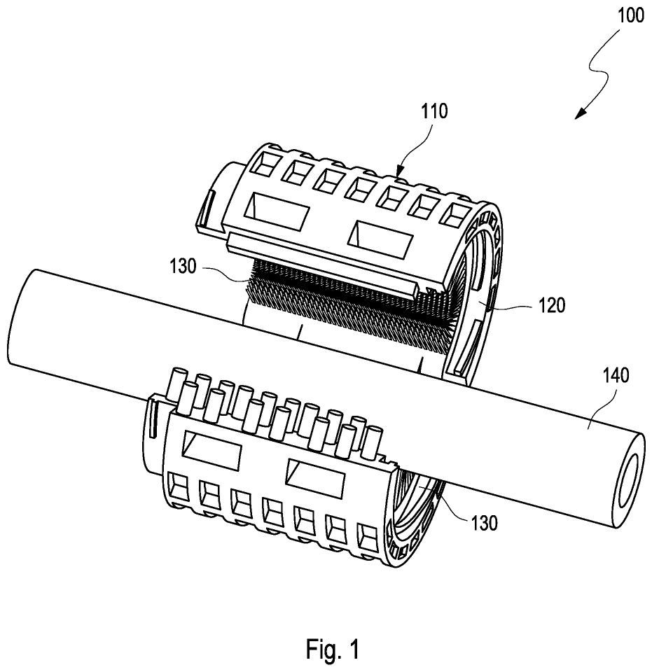

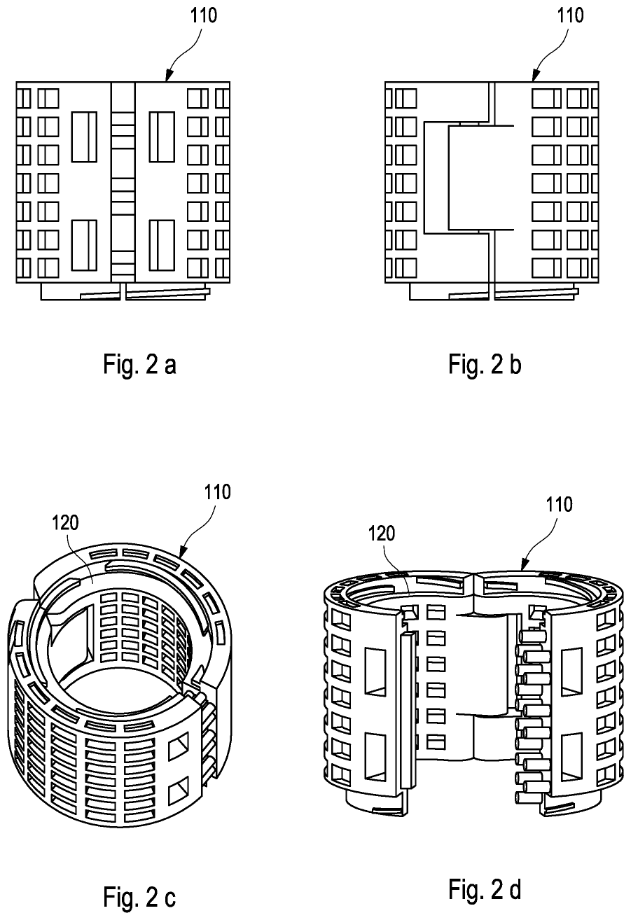

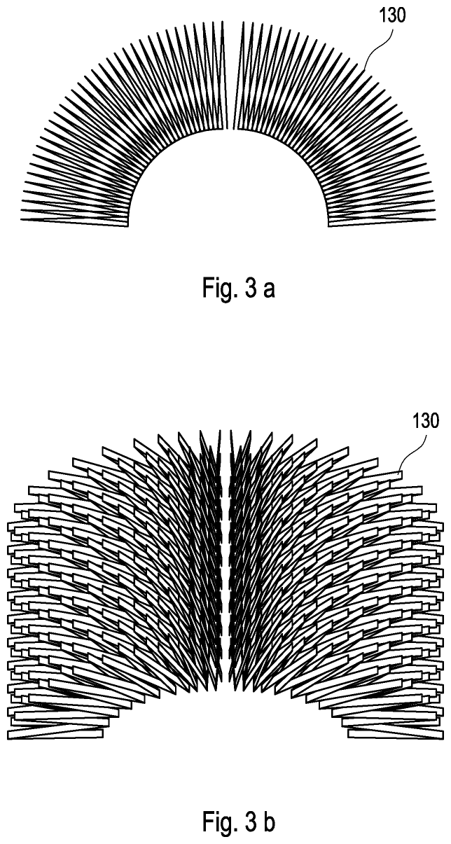

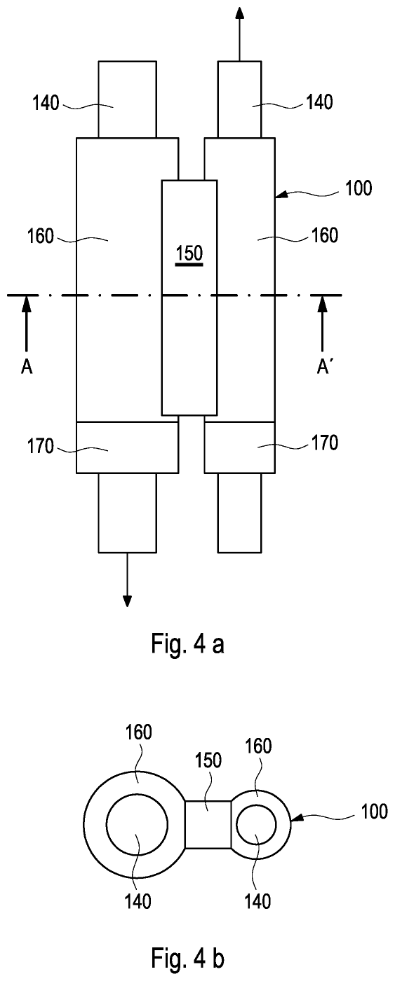

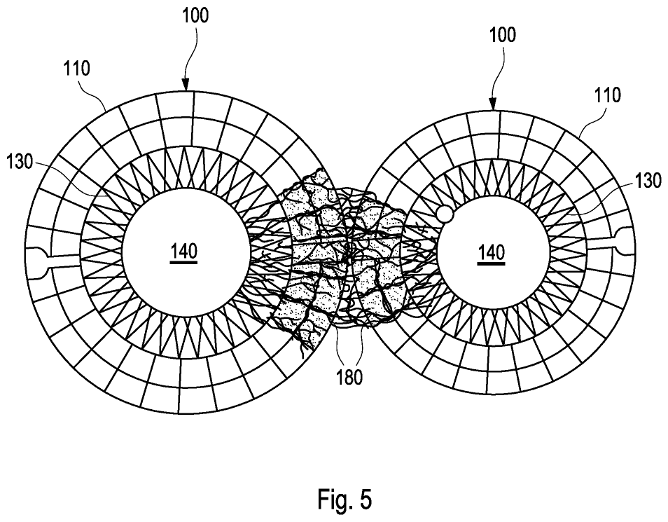

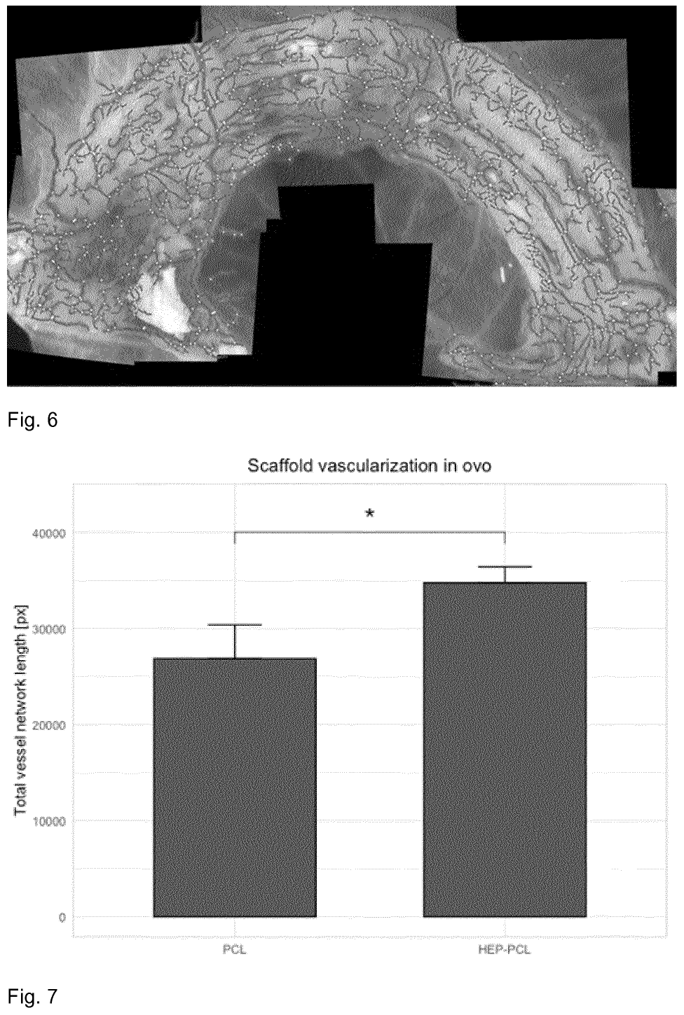

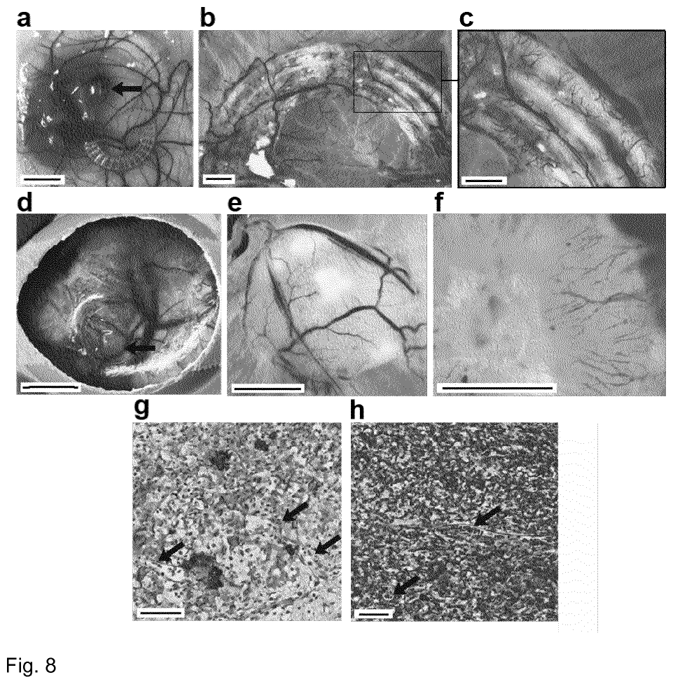

Vascularization of a perivascular implant 100 is crucial for viability and function of effector cells (here: insulin-secreting islets) (Peiris et al., 2014, loc. cit., Smink et al., 2017, loc. cit., Brissova et al., Diabetes 2006; 55:2974-85). The transplantation of the housing component 110 (FIG. 8a-c) and effector matrix 130 (FIG. 8d-f) to the CAM of fertilized chicken eggs was utilized to investigate vascular ingrowth and vessel penetration through the solid PCL channel architecture of housing elements 110 in vivo (FIGS. 6-8). The surface-functionalized PCL had beneficial effects on vascularization, as shown by the significantly enhanced total vessel network length (untreated control: 26856±3502SD [px], Hep-PCL: 34766±1650SD [px], p<0.05) (FIG. 1,2).

Especially in the initial period after implantation, effector and helper cells are faced with hypoxia and diffusion-based supply. We showed that effector cells (here: insulin-secreting pseudoislets) contained in effector matrix 130 (here: bioprinted gelatin methacrylate/alginate/xanthan gum/laminin 411-blended hydrogel mixture) survived this initial period before formation of vascularization. After 9 days of in ovo cultivation, effector matrices 130 were explanted. A dense host-derived vascular network surrounding the effector matrices 130 was observed (FIG. 8e). Stereomicroscopy showed that vessels penetrated the effector matrix structure (FIG. 8f). Subsequent immunohistochemical analysis proved not only extensive peri-islet vascularization but also intra-islet vascularization (here: effector cells) (FIG. 8g, h). Staining with avian anti-CD34 showed capillary sprouting at the periphery and migration of host endothelial cells towards the graft center without additional external mitogenic stimulation.

3D-Bioprinted insulin cells, serving as effector cells in effector matrix 130 components formed pseudoislets in vitro. This pseudoislet formation was also observed after excision of in ovo cultured bioprinted effector matrix 130. Effector matrices 130 remained on the CAM for 9 days supplied with nutrients only by diffusional processes from the CAM and newly formed vascular structures penetrating the effector matrix 130. The CAM assay modeled the initial period after implantation, in which comprehensive vascularization of the perivascular implant 100 is yet to develop. Effector cells survived this initial period. Immunohistochemical staining against insulin demonstrated that effector cells remained functional until explantation (FIG. 8g).

### LITERATURE

Aleksandrowicz et al., ALTEX 2015; 32:143-147
Berg et al., Nat Methods 2019; 16:1226-32
Brissova et al., Diabetes 2006; 55:2974-85;
Gamble et al., Islets 2018; 10:80-94;
Peiris et al., Diabetes 2014; 63:3-11;
Salg et al., J Tissue Eng 2019; 10:1-25;
Salg et al. (2022), J Tissue Eng 13, 20417314221091033, doi: 10.1177/20417314221091033
Schindelin et al., Nat Methods 2012; 9:676-682;
Siddiqui et al., Mol Biotechnol 2018; 60:506-532;
Smink et al., Ann Surg 2017; 266:149-157;
US 2005/209687 A1;
Zhao et al., Int J Cancer 2018; 142:1440-52.

### REFERENCE SIGNS

100 perivascular implant
110 housing
120 receptacle
130 effector matrix
140 tubular bodily structure
150 bridging element
160 fastening element
170 sensor element
180 coating

1-22. (canceled)

23. A perivascular implant (100) comprising:
(i) a housing (110) comprising a receptacle (120) for an effector matrix (130); and
(ii) a fastening element (160) adapted for at least partially embracing a tubular bodily structure (140),
wherein the housing (110), the receptacle (120) for an effector matrix (130), and the fastening element (160) are arranged such that after implantation of the perivascular implant (100) the receptacle (120) for an effector matrix (130) at least partially is in contact with the tubular bodily structure (140) and/or a vasculature originating from the tubular bodily structure (140).

24. The perivascular implant (100) of claim 23, wherein the tubular bodily structure (140) comprises at least one blood vessel.

25. The perivascular implant (100) of claim 23, wherein the tubular bodily structure (140) is a vascular bundle, preferably an epigastric pedicle.

26. The perivascular implant (100) of claim 23, wherein the fastening element (160) is formed by the housing (110) and/or the receptacle (120) for an effector matrix (130).

27. The perivascular implant (100) of claim 23, wherein the housing (110), the receptacle (120) for an effector matrix (130) and the effector matrix (130) form a channel adapted for at least partially embracing the tubular bodily structure (140).

28. The perivascular implant (100) of claim 23, wherein the perivascular implant (100) further comprises an effector matrix (130).

29. The perivascular implant (100) of claim 23, wherein the effector matrix (130) comprises a matrix material and effector cells, preferably wherein the effector matrix (130), and optionally the perivascular implant (100), can be explanted and resected without leaving effector cells within the body of the subject.

30. The perivascular implant (100) of claim 23, wherein the housing (110) is at least partially coated with at least one modulator compound, preferably wherein the modulator compound is heparin and/or a growth factor, preferably basic fibroblast growth factor (bFGF), nerve growth factor (NGF), and/or vascular endothelial growth factor (VEGF).

31. The perivascular implant (100) of claim 23, wherein the perivascular implant (100) is comprised of two essentially half-cylindrical elements together forming a central channel enclosing the tubular bodily structure (140).

32. The perivascular implant (100) of claim 23, wherein the perivascular implant (100) further comprises an effector matrix (130), wherein:
(a) the housing (110), the receptacle (120) for an effector matrix (130) and the effector matrix (130) form a channel adapted for at least partially embracing the tubular bodily structure (140),
(b) the perivascular implant (100) is comprised of two essentially half-cylindrical elements together forming a central channel enclosing the tubular bodily structure (140), and
(c) the tubular bodily structure (140) comprises at least one blood vessel.

33. The perivascular implant (100) of claim 23, wherein the perivascular implant (100) is a first perivascular implant (100) and further comprises a connector permitting interconnection to a second perivascular implant (100).

34. The perivascular implant (100) of claim 33, wherein the tubular bodily structure (140) at least partially embraced by the first perivascular implant (100) is an artery and wherein the tubular structure embraced by the second perivascular implant (100) is a vein.

35. The perivascular implant (100) of claim 33, wherein the first perivascular implant (100) and the second perivascular implant (100) are connected via a bridging element (150).

36. The perivascular implant (100) of claim 23, wherein the perivascular implant (100) comprises at least one sensor element (170), preferably wherein the sensor element (170) comprises a sensor for a blood and/or physiological parameter, preferably wherein the physiological parameter is blood glucose and/or blood insulin.

37. The perivascular implant (100) of claim 28, wherein:
(a) the matrix material comprises gelatin methacrylate blend hydrogel, and/or
(b) the effector cells secrete at least one of insulin, glucagon, a growth hormone, and a cytokine.

38. The perivascular implant (100) of claim 23, wherein the perivascular implant (100) further comprises an effector matrix (130) comprising a matrix material and effector cells, wherein:
(a) the matrix material comprises gelatin methacrylate blend hydrogel,
(b) the effector cells secrete at least one of insulin, glucagon, a growth hormone, and a cytokine,
(c) the housing (110), the receptacle (120) for an effector matrix (130) and the effector matrix (130) form a channel adapted for at least partially embracing said tubular bodily structure (140),
(d) the perivascular implant (100) is comprised of two essentially half-cylindrical elements together forming a central channel enclosing said tubular bodily structure (140), and
(e) the tubular bodily structure (140) comprises at least one blood vessel.

39. A method of treating disease, preferably diabetes, in a subject, the method comprising implanting a perivascular implant (100) according to claim 23 into the subject.

40. The method of claim 39, wherein the perivascular implant (100) comprises effector cells that secrete at least one of insulin, glucagon, a growth hormone, or a cytokine.

41. The method of claim 40, wherein the effector cells are selected from the group consisting of insulinoma cells, human induced pluripotent stem cells, adipose tissue derived stem cells, mesenchymal stem cells, pancreatic precursor cells, and insulin-transfected cells.

42. A kit comprising:
(i) at least two modules of a modular perivascular implant (100) according to claim 23, and/or
(ii) a perivascular implant (100) and at least one of an effector matrix (130), a second perivascular implant (100) and a bridging unit (150).
